(19) United States
(12) Patent Application Publication
(10) Pub. No.: US 2026/0335646 A1
(43) Pub. Date: Oct. 29, 2026

(54) METHODS FOR EPIDURAL SPINAL CORD STIMULATION USING TEMPORARY LEAD PLACEMENT AND FUNCTIONAL MAPPING

(71) Applicant: The United States Government As Represented By The Department of Veterans Affairs, Washington, DC (US)

(72) Inventors: Ashraf Gorgey, Washington, DC (US); Robert Trainer, Washington, DC (US); Lance L. Goetz, Washington, DC (US); Denise Lester, Washington, DC (US); Timothy Lavis, Washington, DC (US)

(21) Appl. No.: 19/551,727

(22) Filed: Feb. 27, 2026

Related U.S. Application Data
(60) Provisional application No. 63/764,753, filed on Feb. 28, 2025.

(51) Int. Cl. A61N 1/36; A61N 1/05

(52) U.S. Cl. CPC A61N 1/36062; A61N 1/0551; A61N 1/36157; A61N 1/36185

(57) ABSTRACT

Methods are disclosed in the field of epidural spinal cord stimulation for spinal cord injury. The methods include temporarily implanting at least one electrical stimulation lead into an epidural space, delivering stimulation while varying electrode configurations and stimulation parameters, and performing spinal segmental mapping based on observed motor responses. Results of the mapping are used to select an epidural implantation site and stimulation configuration for permanent implantation of one or more leads. The permanently implanted system is used to facilitate lower extremity motor function, including during standing or walking activities.

### CROSS-REFERENCE TO RELATED APPLICATIONS

This application claims the benefit of and priority to U.S. Provisional Patent Application No. 63/764,753, filed Feb. 28, 2025, the entire contents of which are incorporated herein by reference.

### BACKGROUND

Spinal cord injury is a major cause of long-term motor impairment, frequently resulting in partial or complete paralysis of the lower extremities. Individuals with such injuries often experience profound limitations in mobility, balance, and postural control, which in turn contribute to secondary medical complications including cardiovascular deconditioning, metabolic dysfunction, musculoskeletal deterioration, autonomic instability, and reduced overall health. These impairments impose substantial personal, social, and economic burdens and significantly affect independence and quality of life.

Despite the severity of motor deficits observed clinically, neural circuitry below the level of injury may remain anatomically intact to varying degrees. However, the functional engagement of these circuits is often disrupted by injury-related changes in neural excitability, synaptic integration, and sensorimotor signaling. As a consequence, voluntary motor commands may fail to effectively recruit spinal motor networks, and residual motor capacity may remain latent or inaccessible during functional activities such as standing or walking.

A further complication arises from the heterogeneity of spinal cord injuries across individuals. Differences in injury level, injury severity, chronicity, and individual neuroanatomy lead to wide variability in motor outcomes and physiological responses. Even among individuals with similar clinical classifications, functional capabilities and responsiveness of spinal circuits can differ substantially. This variability complicates efforts to determine how and where spinal neural activity should be modulated to achieve meaningful motor engagement.

Another challenge is the difficulty of assessing functional motor responsiveness in a manner that reflects task-specific demands. Motor behaviors such as standing, stepping, and walking require coordinated activation of multiple muscle groups across joints and rely on both tonic and dynamic patterns of neural activity. Evaluations performed under static or unloaded conditions may not accurately represent how spinal circuits behave during weight-bearing or task-oriented activities. Consequently, there is uncertainty in predicting whether a given intervention will support functional motor tasks relevant to daily living.

Clinical decision-making is further complicated by the absence of reliable approaches for determining patient-specific suitability for long-term therapeutic intervention. Factors such as discomfort, tolerance, anatomical constraints within the epidural space, and the potential emergence of unforeseen physiological responses can influence whether an individual can safely and effectively undergo prolonged treatment. Without mechanisms to evaluate these factors directly within the spinal environment, long-term decisions may be made without adequate functional information.

Additionally, the process of identifying appropriate anatomical regions associated with desired motor outcomes is inherently complex. The relationship between spinal segmental organization, vertebral anatomy, and functional motor output is not uniform across individuals. Small variations in anatomical positioning can lead to significant differences in motor recruitment patterns, including which muscles are activated, the strength of activation, and whether activation is sustained or transient.

Collectively, these challenges highlight the difficulty of reliably engaging spinal motor networks in a patient-specific manner that supports meaningful motor function. There remains a need for approaches that address the variability, uncertainty, and functional complexity inherent in spinal cord injury, while enabling informed clinical decisions regarding long-term motor rehabilitation strategies.

### SUMMARY

In one aspect, the disclosure relates to a method for implanting and configuring an epidural spinal cord stimulation system for a subject with spinal cord injury. The method includes temporarily implanting at least one electrical stimulation lead into an epidural space of the subject and delivering epidural electrical stimulation through the temporarily implanted lead. While the lead remains temporarily implanted, spinal segmental mapping is performed by varying electrode configurations and stimulation parameters and by assessing motor responses of one or more lower extremity muscles. Based on the mapping, a target epidural location and/or stimulation configuration associated with a desired motor activation pattern is determined. The temporarily implanted lead is then removed, and at least one electrical stimulation lead is permanently implanted into the epidural space at the target location determined from the mapping.

In another aspect, the disclosure relates to a method for selecting an epidural implantation site for permanent spinal cord stimulation. The method includes temporarily placing at least one electrical stimulation lead within an epidural space of a subject and delivering epidural stimulation through the temporarily placed lead using multiple electrode configurations or stimulation parameter sets. Motor responses of the subject's lower extremities are evaluated during stimulation, and epidural locations are mapped to corresponding motor response characteristics. Based on the mapping, an epidural implantation site is selected for permanent placement of a spinal cord stimulation lead.

In a further aspect, the disclosure relates to a method of treating a subject with spinal cord injury. The method includes temporarily implanting at least one epidural stimulation lead into an epidural space of the subject and delivering epidural stimulation through the temporarily implanted lead while varying stimulation parameters to evoke motor responses of the subject's lower extremities. Motor responses are mapped to stimulation parameters and epidural lead locations, after which the temporarily implanted lead is removed. At least one epidural stimulation lead is permanently implanted based on the mapping, and epidural stimulation is delivered through the permanently implanted lead to facilitate lower extremity motor function.

In some embodiments, spinal segmental mapping includes assessing electromyographic (EMG) signals, visible muscle contractions, tonic motor activation patterns, rhythmic motor activity, or combinations thereof. In some embodiments, the mapping is used to distinguish stimulation configurations suitable for standing from configurations suitable for stepping or locomotion. In some embodiments, the mapping is performed in multiple postures or functional conditions and is used to determine whether the subject is a candidate for permanent implantation.

In some embodiments, the permanently implanted stimulation system is operated during standing, walking, or assisted walking activities, including device-assisted gait training. In some embodiments, the mapping identifies stimulation configurations that reduce the stimulation amplitude required to evoke motor responses, improve coordination of muscle activation, or enhance functional motor performance.

Together, these aspects provide methods in which temporary epidural implantation and functional mapping are used to inform patient-specific decisions regarding permanent epidural lead placement and stimulation configuration, thereby enabling tailored treatment strategies for individuals with spinal cord injury.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 shows different phases of implantation. As shown in panel a, the process starts with conducting T2-MRI to verify the location and the size of the injury as well as determining the axial tissue damage as previously highlighted. Panels b1 and b2 highlight the procedure of temporary implantation. The procedure is conducted in a minor procedure room and after local anesthesia, the temporary leads are threaded in the epidural spaces then anchored from outside the body once the anatomical location is verified by fluoroscopy as indicated in panel b2. Panel c shows a representative DXA image of the spinal region depicting leads placement with inter-pulse generator. The eight rectangular blocks on the leads are representative of the electrodes. The leads are seen to be staggered where the left lead covered the midline of the vertebral body of T11 till the upper border of the L1 vertebral body, and the right lead extends from T11-T12 intervertebral disc to the middle portion of the L1 vertebra.

FIG. 2 shows spinal mapping configurations resulting in right leg flexion. Left leg muscles are depicted in black, right leg muscles in gray. Both plots occurred simultaneously but have been separated to better visualize different effects of voluntary effort between legs. At 1.3 mA, bilateral muscle activity was induced. However, when the participant volitionally attempted to flex his right leg, muscle activity decreased in some left leg muscles (VL, RF, HS) while increasing or bursting in right leg muscles (RF, TA, HS, MG). Hz=hertz (stimulation frequency); μs=microseconds; L=left; R=right; VL=vastus lateralis; RF=rectus femoris; TA=tibialis anterior; HS=hamstrings; MG=medial gastrocnemius; sec=seconds; uv=microvolts.

FIG. 3 shows two exemplary recruitment curves at different cathode/anode activation configurations, for left and right legs, detecting activity at the Vastus medialis (VM) muscle of the quadriceps and hamstrings (HM).

FIG. 4 shows an example plot of rhythmic EMG activities of different muscle groups after setting the amplitude at 5.2 and 5.4 mA to be used in practicing stepping and locomotion at higher frequencies during either EAW or overground locomotion. Hz=hertz (stimulation frequency); μs=microseconds; L=left; R=right; VL=vastus lateralis; RF=rectus femoris; TA=tibialis anterior; HS=hamstrings; MG=medial gastrocnemius.

FIG. 5 shows an exemplary electrical stimulation lead that can be used with the disclosed methods.

### DETAILED DESCRIPTION

Embodiments of the present disclosure relate to methods for epidural spinal cord stimulation comprising (i) temporary epidural lead implantation, (ii) functional spinal segmental mapping performed while the lead remains temporarily implanted to determine effective epidural locations and stimulation settings, and (iii) permanent implantation of one or more epidural leads at locations selected based on the mapping. Unless otherwise indicated, steps may be performed in any medically appropriate order, and optional steps may be omitted or repeated, provided that temporary implantation and mapping are used to inform permanent implantation.

### DEFINITIONS

As used herein, unless otherwise specified, terms shall have their ordinary meaning as understood by one of ordinary skill in the relevant art. The singular forms “a,” “an,” and “the” include plural referents unless the context clearly dictates otherwise. The term “comprising” is synonymous with “including,” “containing,” or “characterized by,” and is inclusive and open-ended, permitting the inclusion of additional elements not expressly recited. Similarly, the terms “consisting essentially of” and “consisting of” may be used in other contexts to limit or exclude additional elements, as understood in patent practice. When ranges are recited, it is intended that the range includes all endpoints and intermediate values.

The term “subject” includes a human and a non-human mammal. The term “spinal cord injury” includes traumatic and non-traumatic injury, complete and incomplete injury, and injuries classified under standardized neurological scales. In particular implementations, the subject presents motor-complete injury characterized by absent or minimal voluntary lower extremity movement below the neurological level of injury.

The term “epidural space” refers to the anatomical compartment external to the dura mater. Epidural lead placement may be posterior or posterolateral relative to the dura and spinal cord, and may be performed at thoracic, thoracolumbar, or lumbar vertebral levels to modulate neural circuits associated with lower extremity function.

The term “lead” refers to an elongate, implantable, flexible body that carries a plurality of electrodes (e.g., rings, cylindrical bands, segmented pads, or other conductive members) spaced along a distal region of the flexible body. Leads may be percutaneous leads introduced through a needle, or paddle leads introduced through a laminotomy or laminectomy. In one non-limiting implementation, each lead includes eight electrodes arranged in a linear array; other electrode counts and spacings may be used. In one embodiment, the lead is not a paddle lead introduced through a laminotomy or laminectomy but rather a percutaneous lead which requires relatively minimal surgical intervention and accordingly is preferred in some embodiments over paddle leads.

The term “electrode configuration” refers to a selection of which electrode(s) are assigned as cathode(s), which electrode(s) are assigned as anode(s), and which electrode(s), if any, are inactive. A configuration may be monopolar (e.g., one cathode with a distant anode such as a device case), bipolar (one cathode and one anode on the lead), multipolar (multiple cathodes and/or anodes), and may include field-shaping using adjacent or non-adjacent electrodes.

The term “stimulation parameter” includes stimulation frequency (Hz), pulse duration or pulse width (microseconds), current amplitude (mA) or voltage amplitude (V), burst parameters where applicable, duty cycle, ramp profiles, and inter-pulse intervals. Parameter values may be adjusted to achieve thresholds for activation and to shape tonic and rhythmic motor patterns.

The term “mapping” or “spinal segmental mapping” refers to a systematic evaluation performed with the epidural lead in place in which stimulation is delivered using multiple electrode configurations and/or parameter sets, and a physiological response is assessed to determine which epidural locations and stimulation settings (e.g., electrode configurations and/or stimulation parameters) produce desired effects. Mapping may be performed intraoperatively, postoperatively, across multiple sessions, and/or in multiple postures and task contexts.

The term “motor response” includes any measurable or observable indicator of neuromuscular activation, including visible muscle contraction, joint movement, force production, electromyographic (EMG) signals, changes in reflex excitability, changes in muscle tone, changes in coordinated patterns of activation across multiple muscles, or combinations thereof.

The term “tonic activation pattern” refers to a sustained or quasi-sustained increase in activation of one or more muscles or muscle groups over a time window that is longer than a single stimulus artifact and that is suitable for generating or supporting static or slow-varying joint torques. Tonic activation patterns are typically characterized by (i) relatively continuous EMG envelope elevation, (ii) maintenance of extensor or flexor activity during a maintained posture (e.g., standing), and/or (iii) consistent joint torque or limb position over multiple seconds. Tonic patterns may be produced by stimulation settings that favor recruitment of motor pools and interneuronal networks that maintain extensor tone, and may be tuned by adjusting pulse width, amplitude, frequency, and/or field shape.

The term “rhythmic motor activity” or “rhythmic activation pattern” refers to a time-varying pattern that exhibits periodicity or alternation over time, such as alternating activation of antagonistic muscles (e.g., tibialis anterior and gastrocnemius) and/or alternating activity between left and right limbs. Rhythmic patterns may be used to facilitate stepping-like or locomotor-like movement and may be characterized by repeated bursts in EMG with relatively consistent cycle duration, phase relationships, and burst timing.

The term “recruitment curve” refers to a functional relationship between a stimulation intensity variable (e.g., amplitude) and a response magnitude variable (e.g., EMG peak-to-peak amplitude, RMS envelope amplitude, or joint torque). Recruitment curves may be used to determine thresholds, dynamic range, saturation behavior, and comparative selectivity across electrode configurations.

The term “lumbosacral enlargement” refers to spinal cord segments that contain neural circuitry associated with lower extremity motor control. In many subjects, these segments correspond anatomically to vertebral levels in the lower thoracic to upper lumbar region, and epidural stimulation sites may be selected accordingly to modulate lower extremity motor pools and spinal networks.

The term “temporary implantation” refers to placement of an epidural lead intended to remain in place for a limited time (e.g., hours to days) sufficient to conduct mapping and subject evaluation. Temporary implantation may include externalized lead connectors, external anchoring, and removal without leaving a chronic implant in place.

The term “permanent implantation” refers to implantation of one or more epidural leads intended for chronic use, typically coupled to an implantable pulse generator (IPG) implanted in a subcutaneous pocket and connected by tunneled extensions. Permanent implantation may include lead anchoring strategies to reduce migration.

The term “implantable pulse generator” refers to an implantable device including power storage and pulse generation electronics configured to deliver programmable electrical stimulation waveforms to the implanted lead(s). The IPG may communicate with an external programmer and/or patient controller to adjust configurations and parameters.

### Method of Implanting and Configuring Epidural Spinal Cord Stimulation System

In one embodiment, at least one epidural lead is temporarily implanted percutaneously. A percutaneous approach may include positioning the subject prone, prepping and draping the surgical field, administering local anesthetic and/or sedation as clinically appropriate, and accessing the epidural space using one or more epidural needles. Epidural access may be achieved using a loss-of-resistance technique, in which advancement of the needle through ligamentous tissues is monitored until a characteristic change in resistance indicates entry into the epidural space.

Imaging guidance may be used to confirm needle trajectory and epidural placement. Fluoroscopy may be used to visualize bony landmarks and radiopaque markers on the lead, enabling placement relative to targeted vertebral levels. Leads may be advanced cephalad or caudad to a target region associated with lower extremity motor function. In a non-limiting implementation, two leads are advanced to overlie a region spanning approximately T10 through L2 spinal segments or vertebral levels associated with the lumbosacral enlargement.

Temporary implantation may include bilateral lead placement, with leads guided to either side of the midline to modulate left and right motor pools. Leads may be positioned in a staggered or offset arrangement to provide coverage across multiple segmental levels and to enable lateralized field shaping. Temporary leads may be externalized and secured to the skin using adhesive dressings, sutures, tape, medical-grade adhesive, and/or anchoring devices to limit migration during the temporary implantation period.

After temporary implantation, stimulation is delivered through the lead to verify functional placement and to collect mapping data. Stimulation may be delivered using constant-current or constant-voltage pulses. Pulses may be rectangular, biphasic, or charge-balanced waveforms. In some implementations, charge balancing is used to reduce net DC and minimize tissue irritation. Stimulation may be administered while the subject is at rest (e.g., supine) and/or during tasks (e.g., attempted voluntary movement, standing, stepping). Clinically appropriate safety monitoring may include hemodynamic monitoring, assessment for discomfort, and assessment for autonomic dysreflexia risk, particularly in subjects with higher-level spinal cord injury.

Spinal segmental mapping may be conducted over one or more sessions while the lead remains temporarily implanted. Mapping includes systematically varying electrode configuration and/or stimulation parameters and assessing the resulting physiological responses. Electrode configuration variation may include: selecting different individual electrodes or groups of electrodes as cathodes; selecting different electrodes as anodes; selecting return pathways that shape the electric field; and selecting inactive electrodes. Configurations may be compared to determine selectivity for particular muscles or movements. A configuration may include up to several electrodes per channel to create wide-field activation (e.g., larger field coverage) or narrow-field activation (e.g., more localized field shaping).

Parameter variation may include varying one or more of: (i) frequency, (ii) pulse duration, and (iii) amplitude. In one implementation, mapping includes testing frequencies such as approximately (e.g., within 5% of) 2 Hz, 15 Hz, and 30 Hz, and pulse durations ranging from approximately 250 microseconds to approximately 1000 microseconds. Amplitude may be varied across a range sufficient to span subthreshold to suprathreshold responses, for example from approximately 1 mA to approximately 10 mA during recruitment curve development, with the objective of using a minimum current necessary to evoke the targeted motor response.

Physiological assessment during mapping may include direct observation of visible muscle contractions and/or joint movement, and/or measurement of EMG signals from one or more lower extremity muscles. EMG assessment may include placement of surface electrodes over selected muscles, acquisition of signals using differential amplification, filtering, and quantification using measures such as RMS envelope, peak amplitude, onset latency, burst duration, and inter-muscle phase relationships.

Mapping may include instructing the subject to attempt voluntary movements with stimulation off and on, enabling determination of how stimulation changes the ability to initiate and sustain movement. Non-limiting movement prompts include toe movement, ankle dorsiflexion, hip flexion, knee extension, and coordinated leg flexion-extension. Mapping may be performed in multiple postures. A supine posture may be used for initial recruitment characterization and to reduce confounds of weight bearing. Upright or standing postures may be used to identify configurations that support extensor tone for standing. Mapping may also be performed during stepping-like tasks or assisted locomotion to identify rhythmic patterns. Mapping may be repeated across multiple days during the temporary implantation window to assess stability, to reduce day-to-day variability, and to determine whether responses change as stimulation is adjusted or as the subject adapts.

In some embodiments, spinal segmental mapping comprises recording electromyographic (EMG) signals from a plurality of lower extremity muscles. EMG may be recorded bilaterally from muscles including, for example, tibialis anterior, medial gastrocnemius, soleus, hamstrings, quadriceps (including vastus lateralis and rectus femoris), and gluteus maximus. EMG electrodes may be placed over motor points with standardized skin preparation. Signal acquisition may include bandpass filtering to reduce motion artifacts and electrical interference, and additional processing to reduce stimulation artifacts. Metrics may include RMS envelopes, peak amplitudes during defined time windows, and coordination metrics such as co-contraction indices or phase offsets between muscles.

EMG may be used to quantify tonic activation patterns (e.g., sustained elevated EMG during a maintained posture) and rhythmic activation patterns (e.g., repeated bursts with identifiable cycle timing). EMG may also be used to generate recruitment curves by measuring response amplitude as a function of stimulation amplitude.

In some embodiments, mapping identifies configurations that produce tonic motor activation associated with leg flexion and/or knee extension. For leg flexion, tonic activation may be characterized by sustained activation of hamstrings and/or hip flexors and associated joint movement or maintained flexed posture. For knee extension, tonic activation may be characterized by sustained activation of quadriceps muscles including vastus lateralis and rectus femoris and associated extension torque or maintained knee extension. Mapping may identify different amplitude thresholds for different movements, enabling program selection that preferentially recruits desired muscle groups at lower amplitude.

Tonic activation patterns may be further refined by adjusting pulse width and frequency to stabilize muscle tone without inducing uncomfortable sensations or excessive fatigue. Where tonic activation is intended for standing, mapping may emphasize extensor muscle groups and may include testing in upright posture to confirm functional support for weight bearing.

In some embodiments, mapping identifies configurations that produce rhythmic motor activity associated with stepping or locomotion. Rhythmic activity may be characterized by periodic EMG bursts in flexor and extensor muscles, including alternating activation of dorsiflexors (e.g., tibialis anterior) and plantarflexors (e.g., gastrocnemius/soleus). Rhythmic patterns may be evoked at low frequencies during mapping to reveal timing and coordination, and may be adapted to higher-frequency stimulation or combined with task-specific cues to support stepping. Rhythmic mapping may be performed during assisted stepping conditions such as parallel bars, a standing frame, body-weight support, or exoskeleton-assisted walking, to determine whether stimulation supports coordinated limb advancement and stance stability.

In one embodiment, mapping results are used to determine a target epidural location and/or configuration for subsequent permanent implantation. The target may be determined by selecting the location and configuration that produces a desired motor activation pattern with acceptable thresholds, selectivity, and tolerability. Desired motor activation patterns may include tonic activation patterns suitable for standing (e.g., sustained activation of extensor muscles such as quadriceps and plantarflexors) and/or rhythmic activation patterns suitable for stepping (e.g., periodic alternation between dorsiflexors and plantarflexors). In one non-limiting example, mapping identifies stimulation settings that evoke tonic leg flexion and knee extension at different amplitude thresholds, and identifies settings that evoke rhythmic EMG activity at low frequency that can be scaled for stepping training at higher frequencies.

Mapping may include generating recruitment curves for multiple configurations and selecting configurations that provide desired activation within a safe and controllable amplitude window. Mapping may also include categorizing configurations as wide-field versus narrow-field based on the distribution of activation across muscles and the shape of the recruitment curves.

In some embodiments, the target epidural location is selected to correspond to spinal segments associated with the lumbosacral enlargement. The mapping procedure may be used to confirm that stimulation at a given vertebral level produces robust lower extremity motor responses. Leads may be positioned to span multiple vertebral levels so that electrode combinations can steer stimulation to the most effective segmental targets identified during mapping. Selection may involve choosing left-right symmetry or deliberate asymmetry depending on whether unilateral or bilateral activation is desired.

In one embodiment, a determination to proceed with permanent implantation is made based on mapping results and clinical evaluation. The determination may consider whether effective motor responses can be elicited, whether stimulation is tolerated, and whether the epidural space anatomy allows stable lead placement. After mapping and evaluation, the temporary lead is removed. Removal may include withdrawing the lead through the percutaneous entry path, applying hemostasis, and dressing the site. Where temporary leads are externalized, connectors may be disconnected from external equipment prior to removal.

In one embodiment, one or more leads are permanently implanted into the epidural space at a location selected based on temporary mapping. Permanent implantation may use the same or similar epidural access techniques (e.g., loss-of-resistance) and imaging guidance (e.g., fluoroscopy) as the temporary procedure. Permanent implantation may include placing two leads, each carrying a plurality of electrodes, and positioning them in a staggered configuration spanning multiple vertebral levels. Staggering may increase the available set of electrode combinations for field shaping and may provide more robust coverage for both tonic and rhythmic programs.

In some embodiments, permanently implanting comprises implanting a plurality of leads in a staggered configuration spanning multiple vertebral levels. Staggering may include offsetting the distal electrode arrays such that electrodes on one lead are longitudinally shifted relative to electrodes on the other lead. This arrangement can increase the diversity of electrode pairings and can provide more granular control over rostrocaudal field placement. A staggered arrangement may also provide redundancy such that if one electrode or contact exhibits higher impedance or reduced effectiveness, alternative contacts remain available for programming.

An implantable pulse generator (IPG) is implanted in a subcutaneous pocket (e.g., in a lower back or buttock region) and is electrically coupled to the permanently implanted lead(s) via tunneled extensions. The IPG is programmed with stimulation configurations and parameter sets determined from mapping, and may store multiple programs for different postures and activities. In some embodiments, an IPG is coupled to the permanently implanted lead(s) and is programmed to deliver stimulation according to configurations and parameter sets determined during mapping. The IPG may store multiple programs corresponding to different postures or activities, such as a standing program emphasizing tonic extensor activation and a stepping program emphasizing rhythmic activity. The IPG may allow clinician programming and patient-controlled selection of stored programs, subject to clinician-set limits. The IPG may implement safety features such as amplitude limits, ramp-up/ramp-down, impedance checks, charge balancing, and session timers.

After permanent implantation, mapping may be repeated to fine-tune stimulation settings, to confirm that chronic lead position reproduces desired responses, and to adapt programs to standing, stepping, and locomotion tasks.

### Method of Selecting an Epidural Implantation Site

In further embodiments, a temporary epidural lead is placed within the epidural space and used primarily as a diagnostic and selection tool to choose an epidural implantation site for permanent stimulation. The temporary lead may be positioned at one or more candidate levels and may be repositioned during the selection process.

Stimulation is delivered using a plurality of electrode configurations and parameter sets. The plurality may be defined by systematically iterating through a library of cathode-anode assignments and parameter sweeps. The library may be constrained to clinically safe ranges and may be tailored to expected segmental targets associated with lower extremity motor pools.

Motor response evaluation may include qualitative observations (e.g., presence/absence of contraction, joint movement direction) and quantitative measures (e.g., EMG amplitude, torque, timing). Mapping epidural locations to motor response characteristics may be recorded in a mapping log that associates each tested configuration with observed responses, thresholds, and tolerability. Response characteristics may include: activation threshold, selectivity for specific muscle groups, symmetry between limbs, ability to produce extensor tone for standing, ability to produce alternating bursts for stepping, and robustness across repeated trials.

In certain embodiments, motor responses are evaluated by observing visible muscle contractions in paralyzed lower extremity muscles. Visible contractions may be confirmed by palpation, observation of joint movement, or observation of muscle belly activation. Observations may be recorded for each configuration, along with thresholds and qualitative notes regarding contraction strength and distribution.

In some embodiments, motor response evaluation includes analyzing EMG recruitment curves. Recruitment curves may be generated by stepping amplitude through a set of values while holding frequency and pulse duration fixed, and recording EMG amplitude. Curves may be normalized to a maximum observed amplitude for comparison across sessions. Curve features such as threshold, slope, and saturation may be used to select sites and configurations that provide controllable activation.

In some embodiments, mapping explicitly distinguishes tonic motor responses from rhythmic motor responses. Tonic responses may be scored based on sustained EMG elevation and maintained joint torque, whereas rhythmic responses may be scored based on burst periodicity, alternation, and stability of cycle timing. Separate programs may be developed for tonic standing versus rhythmic stepping, and site selection may favor locations capable of supporting both or may prioritize one depending on the intended therapy.

In some embodiments, temporary placement is maintained for multiple days to perform repeated mapping sessions. Multi-day mapping may allow evaluation under different conditions, repetition to ensure consistency, and iterative refinement of programs. During the multi-day period, external anchoring may be used to limit lead migration, and mapping sessions may be scheduled over consecutive days.

In certain embodiments, site selection includes determining whether the subject is a candidate for permanent implantation. Candidacy may be determined based on whether effective motor patterns are achieved at tolerable stimulation levels, whether the temporary implantation can be maintained without complications, whether the subject can tolerate the stimulation and temporary lead period, and whether anatomical constraints allow stable placement.

Based on the mapping, an epidural implantation site is selected for permanent placement. The selected site may correspond to a vertebral level, an intervertebral disc space reference, or a relative position with respect to the midline. Selection may also include selecting a lead orientation and lateral offset. Where two leads are intended for chronic implantation, site selection may include selecting bilateral sites and an offset between leads. In some embodiments, selection includes choosing between wide-field configurations that recruit multiple muscles versus narrow-field configurations that target a specific muscle group, depending on the intended therapeutic objective.

In some embodiments, after selecting a site based on temporary mapping, a permanent lead is implanted at the selected site. Permanent implantation may include repeating imaging-guided placement to replicate the selected location and orientation, and may include confirming placement by re-testing motor responses and/or repeating mapping post-implantation.

### Treatment Method Using Sequential Temporary and Permanent Epidural Stimulation

In some embodiments, the method is practiced as a treatment method for a subject with spinal cord injury. Treatment includes temporary implantation, parameter variation to evoke motor responses, mapping of responses to parameters and locations, removal of the temporary lead, permanent implantation based on mapping, and delivery of stimulation through the permanently implanted lead to facilitate lower extremity motor function.

Facilitation of lower extremity motor function may include enabling or enhancing standing, stepping, assisted walking, improved muscle activation coordination, improved voluntary contribution during assisted gait, and/or improved motor output during task-specific training.

In treatment embodiments, mapping serves to identify stimulation programs that can be used therapeutically. Mapping links electrode configurations and parameters to functional outcomes such as extensor support for standing and rhythmic bursts for stepping. Mapping may also be used to establish safe operating limits for amplitude and pulse width during therapy sessions and to identify programs tailored to different tasks.

In some embodiments, mapping identifies stimulation parameters and configurations that reduce the amplitude required to evoke motor responses. For example, selecting an electrode configuration that better aligns the field with targeted neural structures may lower threshold compared to another configuration. Increasing pulse duration may also reduce amplitude requirements for a given response, while potentially affecting comfort and selectivity. Mapping may therefore involve balancing amplitude, pulse width, and configuration to achieve desired responses at lower current levels. Reduced amplitude requirements may provide expanded safety margin, reduced energy consumption, and improved comfort during chronic use.

In some embodiments, mapping is performed in at least a supine position and a standing position. Supine mapping may be used to generate recruitment curves and to identify initial configurations without weight-bearing demands. Standing mapping may be used to confirm that selected configurations provide functional extensor support and to adjust frequency and amplitude for posture-dependent changes in excitability. In some embodiments, mapping is further performed during stepping practice, including assisted stepping, to confirm rhythmic patterns and functional coordination.

After permanent implantation, stimulation is delivered in accordance with programs identified and refined through mapping. Stimulation may be delivered continuously during a therapy session, intermittently, or in response to user input. The subject may use an external controller to turn stimulation on or off and to select among clinician-prescribed programs. Stimulation may be combined with external supports and therapy modalities, including assisted standing, stepping, overground walking, and device-assisted gait training.

In some embodiments, stimulation through the permanently implanted lead is delivered during standing or walking training. A standing program may emphasize tonic extensor activation to stabilize knees and hips and to support weight bearing. A walking program may include tonic components for stance stability and rhythmic components for swing initiation. Training may include repeated sit-to-stand transitions, static standing holds, and stepping practice. Safety monitoring may include blood pressure monitoring during upright training and rest periods as needed.

In some embodiments, stimulation is delivered during exoskeleton-assisted walking. An exoskeleton may provide controlled joint trajectories and adjustable assistance. Stimulation may be used to increase active participation by the subject, reduce required exoskeleton assistance, and promote coordinated muscle activation. Programs may be selected to align with gait phases, such as promoting dorsiflexor activation during swing and extensor activation during stance. Assistance may be reduced progressively based on performance metrics such as proportion of steps completed without cueing or without device-driven movement, and stimulation settings may be adjusted accordingly.

In some embodiments, the treatment improves voluntary motor control, assisted stepping performance, or both. Improvements may be measured by increased ability to initiate or sustain voluntary movement with stimulation, increased number of unassisted or less-assisted steps during assisted gait, improved EMG coordination, improved walking speed during standardized tests, and/or improved ability to maintain standing with reduced external support. Improvements may be supported by combining stimulation with task-specific training that leverages the mapped tonic and rhythmic programs.

### Exemplary Embodiments Related to Implantation, Stimulation, and Mapping

In a non-limiting implementation consistent with the disclosed embodiments, temporary implantation uses two 14-gauge epidural needles and fluoroscopic guidance to place two eight-electrode percutaneous leads in a posterior epidural position. Lead placement may be confirmed by observing visible motor contractions during test stimulation. Temporary leads may be secured externally using tape and adhesive and maintained for multiple days of mapping sessions.

In some embodiments, two percutaneous epidural leads are utilized during the temporary implantation phase. Each lead is threaded into the epidural space through a corresponding epidural needle, preferably a 14-gauge needle, using a loss-of-resistance technique under live fluoroscopic guidance. The leads are advanced to opposing sides of the midline to provide bilateral coverage of the lumbosacral spinal segments and to permit testing of multiple stimulation configurations in real time. This percutaneous, needle-based implantation approach is minimally invasive relative to surgical paddle-lead implantation techniques that require laminectomy and open surgical exposure of the epidural space. By avoiding laminectomy and permanent surgical fixation during the evaluation phase, the temporary implantation procedure reduces procedural risk, limits tissue disruption, and enables same-day discharge of participants following implantation, while still allowing confirmation of correct lead placement and functional activation of target spinal segments prior to permanent implantation.

Although the embodiments described herein emphasize stimulation of the lumbosacral spinal segments to facilitate lower extremity motor function, the methods and systems of the invention are not limited to lower extremity applications. The same temporary-to-permanent percutaneous implantation approach may be applied to epidural stimulation targeting cervical and/or upper thoracic spinal segments to facilitate activation, modulation, or restoration of upper extremity motor function, sensory function, or combined sensorimotor function, including but not limited to shoulder, arm, wrist, and hand movements. In such embodiments, temporary percutaneous leads are implanted in the epidural space at spinal levels corresponding to upper extremity innervation to confirm appropriate segmental activation, stimulation tolerance, and functional response prior to permanent implantation. Accordingly, the invention encompasses neuromodulatory therapies directed to upper extremities, lower extremities, or both, and is not restricted to a particular anatomical region or functional modality.

In a non-limiting implementation, mapping includes testing multiple configurations and parameter sweeps, including frequencies on the order of a few Hz to tens of Hz, pulse durations on the order of hundreds of microseconds, and amplitudes on the order of a few mA, to determine configurations that produce tonic and rhythmic patterns. Recruitment curves may be generated at low frequency with amplitude sweeps from low to higher current values and used to compare wide-field and narrow-field configurations.

In a non-limiting implementation, permanent implantation includes implantation of two eight-electrode leads and an implantable pulse generator in a subcutaneous pocket, with leads tunneled to the pocket and connected to the generator. The leads may be positioned in a staggered configuration spanning multiple vertebral levels to provide a broad programmable field.

In a non-limiting implementation, stimulation through the permanently implanted system is used during rehabilitative activities including standing and assisted gait. Rehabilitative activities may include exoskeleton-assisted walking with progressive reduction of device assistance, and task-specific training such as sit-to-stand and trunk control exercises using a walker or standing frame.

The methods described herein are directed to a technological improvement in neuromodulation systems and implantation techniques, and not to the mere evaluation or analysis of physiological data. The disclosed methods integrate specific hardware components, defined procedural steps, and real-time physiological feedback to solve concrete technical problems associated with epidural stimulation therapy. In particular, the methods improve the functioning, reliability, and safety of epidural stimulation systems by introducing a structured, temporary-to-permanent implantation workflow that enables accurate lead placement and stimulation configuration prior to irreversible surgical implantation.

A central technical problem addressed by the methods is that anatomical variability within the epidural space, combined with uncertainty in stimulation responsiveness, can result in ineffective or suboptimal permanent lead placement when implantation is performed without prior functional verification. The methods resolve this problem through temporary percutaneous implantation of epidural leads using fluoroscopically guided needle access, for instance, followed by active stimulation, spinal segmental mapping, and physiological response verification. These steps involve precise manipulation of physical components within the epidural space, controlled delivery of electrical signals, and iterative adjustment of stimulation parameters based on observed motor and electrophysiological outcomes. The process thereby produces a physical change in system configuration that directly improves the performance of the neuromodulation device.

The spinal mapping procedures described herein further constitute a technical improvement by enabling deterministic configuration of stimulation parameters that are tailored to individual anatomy and neural responsiveness. Stimulation frequency, amplitude, pulse duration, electrode polarity, and spatial configuration are actively modified during the temporary implantation phase to achieve specific, reproducible motor activation patterns. This approach improves the functional operation of the stimulation system itself. The resulting configurations are then transferred to the permanent implantation, thereby enhancing the accuracy, predictability, and therapeutic effectiveness of the implanted system.

The described methods are tied to physical devices, invasive medical procedures, and real-time interaction with neural tissue. Embodiments of the techniques involve coordinated operation of epidural leads, fluoroscopic imaging systems, stimulation controllers, and physiological monitoring equipment, all functioning together to achieve a tangible therapeutic outcome. The temporary implantation phase directly informs and modifies the subsequent permanent implantation process, resulting in a concrete transformation of both the medical device configuration and the subject's neuromodulatory treatment pathway.

In some embodiments, by reducing reliance on laminectomy-based paddle lead implantation and enabling minimally invasive, same-day procedures during the evaluation phase, the methods further improve the technical efficiency and safety of implantation workflows. The disclosed methods reduce surgical burden, mitigate the risk of unnecessary permanent implantation, and improve the likelihood that permanent leads are placed at anatomically and functionally optimal locations. These improvements arise from the technical structure and operation of the system rather than generic data processing.

Accordingly, the methods represent a practical application of neuromodulation technology that improves the operation of implantable stimulation systems, enhances the precision of lead placement, and enables reliable restoration of motor and neural function through defined technical means. The disclosed subject matter therefore constitutes a concrete technological solution to longstanding technical challenges in spinal cord stimulation and neurorehabilitation.

### Electrical Stimulation Lead

The electrical stimulation lead is an elongate implantable medical device configured to deliver electrical stimulation from a pulse generator or external stimulator to neural tissue within an epidural space. The electrical stimulation lead serves as the physical interface between the stimulation source and spinal neural structures, and is suitable for use in both temporary implantation during mapping and permanent implantation for chronic therapy.

In epidural spinal cord stimulation applications, the electrical stimulation lead is dimensioned and configured for placement within the posterior epidural space, external to the dura mater. The lead may be positioned such that a distal electrode array overlies spinal cord segments and associated dorsal roots corresponding to targeted motor or autonomic functions, including but not limited to lower extremity motor control.

In one implementation, the electrical stimulation lead is a percutaneous cylindrical lead introducible through an epidural needle. In another implementation, the electrical stimulation lead is a paddle-style lead introducible through a laminotomy or laminectomy. In other embodiments, paddle-style leads are less preferred, and the methods do not involve the use of any paddle-style lead in either a temporary or permanent capacity. The claimed methods are compatible with either lead type, however. Percutaneous leads are particularly well suited for temporary implantation and mapping, while paddle leads may be selected for permanent implantation where increased positional stability or expanded field shaping is desired.

Structurally, the electrical stimulation lead includes an elongate lead body formed of one or more biocompatible insulating polymers. The lead body encloses internal electrical conductors that electrically couple a plurality of distal electrodes to proximal terminal contacts. Conductors may be filar wires, coiled conductors, braided conductors, or combinations thereof, and may be arranged within one or more lumens. In some embodiments, a central lumen is provided to receive a stylet or stiffening element during implantation.

The distal portion of the lead includes an electrode array comprising a plurality of electrically conductive electrodes spaced along the lead body. Electrodes may be ring electrodes, partial-ring electrodes, segmented electrodes, or pad electrodes, and may be formed of platinum, platinum-iridium alloy, or other biocompatible conductive materials. Electrode spacing and geometry may be selected to provide desired rostrocaudal coverage and to enable field shaping through selective activation of electrodes.

The proximal portion of the lead includes a connector portion having a plurality of terminal contacts configured to mate with a header of an implantable pulse generator, an extension, or an external trial stimulator. Each terminal contact is electrically coupled to a corresponding electrode in the distal electrode array, enabling independent selection of electrode configurations during stimulation.

Radiopaque features may be incorporated into the lead, including radiopaque electrode structures, marker bands, or radiopaque polymers, to facilitate visualization under fluoroscopy during implantation. An atraumatic distal tip may be provided to facilitate advancement within the epidural space while reducing tissue trauma.

Optional strain relief regions, anchoring sleeves, or suture features may be included to reduce mechanical stress and to limit migration during permanent implantation. During temporary implantation, the lead may be externalized and secured using external dressings or adhesive fixation.

For temporary implantation, the lead may be introduced percutaneously using an epidural needle and positioned under fluoroscopic guidance. After confirming placement, stimulation is delivered through the lead to perform spinal segmental mapping, including variation of electrode configurations and stimulation parameters to assess motor responses. After mapping, the temporary lead is removed.

For permanent implantation, one or more leads are implanted at epidural locations selected based on the mapping performed during temporary implantation. The lead or leads are tunneled to a subcutaneous pocket and electrically coupled to an implantable pulse generator. Stimulation programs identified during mapping are stored in the pulse generator and used during therapeutic activities such as standing, stepping, or assisted walking.

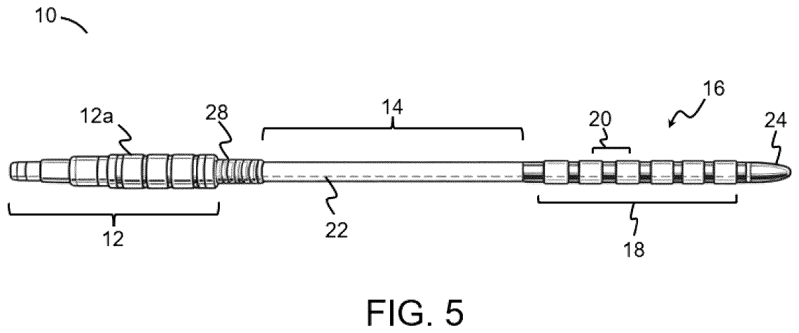

FIG. 5 illustrates a non-limiting example of an epidural electrical stimulation lead suitable for use in the described embodiments. As shown in FIG. 5, an electrical stimulation lead 10 includes a proximal connector portion 12 configured to interface with a pulse generator or external stimulator. The connector portion 12 includes a plurality of terminal contacts 12a, each electrically coupled to a corresponding electrode in a distal electrode array.

An elongate lead body 14 extends distally from the connector portion 12 and provides mechanical flexibility and electrical insulation for internal conductors or internal lumen 22. A strain relief region 28 may be provided adjacent the connector portion to reduce bending stress. The distal portion of the lead includes an electrode array 16 comprising a plurality of electrodes 18 spaced along the lead body with a defined electrode pitch 20. The electrodes 18 are configured to deliver epidural stimulation when selected as cathodes or anodes in a stimulation configuration.

The lead terminates distally in an atraumatic tip 24 to facilitate advancement within the epidural space. One or more radiopaque markers may be provided to enable fluoroscopic visualization of lead position. An optional anchoring sleeve may be positioned along the lead body to reduce migration in permanent implantation. A variety of other types of electrical stimulation leads may be used, and the methods described are not limited to any particular lead embodiment.

### EXAMPLES

The following examples further illustrate this disclosure. The scope of the disclosure and claims is not limited by the scope of the following examples.

### Example 1: Trial Design, Participant Eligibility, Baseline Evaluations, and Randomization

This Example describes the clinical trial design, participant flow, baseline evaluations, and randomization procedures implemented for a twelve-month rehabilitation program combining exoskeleton-assisted walking (EAW), implanted spinal cord epidural stimulation (ES), and resistance training (RT), including a delayed-entry control arm.

Study arms and schedule: Twenty participants were randomized into either (i) EAW+ES+RT (n=10) or (ii) EAW+delayed ES+noRT (n=10) for an intervention period exceeding twelve months. The overall schedule included three measurement weeks (baseline, post-intervention 1, post-intervention 2), a two-week implantation period, and twelve months of training. Measurements were conducted approximately six months apart.

Participant criteria: Participants included male and female individuals aged 18-60 years with traumatic motor-complete spinal cord injury (AIS A or AIS B), at least two years post-injury, with a level of injury below C5.

Screening and baseline assessments: After informed consent, a physiatrist board certified in spinal cord injury medicine performed a full physical examination including neurological assessment and AIS examination. Baseline measurements included exoskeleton fitting, 10-meter EAW speed, number of unassisted steps during EAW walking, EMG pattern from sitting, standing, and walking, H-reflex testing, body composition assessment using anthropometry and dual-energy X-ray absorptiometry (DXA), metabolic profiling, and urodynamic testing.

Randomization and allocation procedure: Randomization was conducted following baseline assessment using the n-query computer program. Stratified randomization was considered based on level of injury and time since injury. Each participant was assigned a sequential ID code (0881, 0882, 0883, etc.). Using a random pattern generator (n=0881-08820), participant numbers were evenly randomized into the two groups. Table 1 provides the disclosed example allocation and order in groups (including control designations).

TABLE 1
In the randomization column, 1: EAW + ES + RT, 0: EAW +
delayed-ES + noRT. In the order column, C: control.

| | | | Order in |
|---|---|---|---|
| Subject ID | Randomization | Assignment | the group |
| 0881 | 1 | EAW + ES + RT | 1 |
| 0882 | 1 | EAW + ES + RT | 2 |
| 0883 | 0 | EAW + delayed-ES + noRT | 1C |
| 0884 | 1 | EAW + ES + RT | 3 |
| 0885 | 0 | EAW + delayed-ES + noRT | 2C |
| 0886 | 1 | EAW + ES + RT | 4 |
| 0887 | 1 | EAW + ES + RT | 5 |
| 0888 | 0 | EAW + delayed-ES + noRT | 3C |
| 0889 | 0 | EAW + delayed-ES + noRT | 4C |
| 08810 | 0 | EAW + delayed-ES + noRT | 5C |
| 08811 | 0 | EAW + delayed-ES + noRT | 6C |
| 08812 | 0 | EAW + delayed-ES + noRT | 7C |
| 08813 | 0 | EAW + delayed-ES + noRT | 8C |
| 08814 | 0 | EAW + delayed-ES + noRT | 9C |
| 08815 | 1 | EAW + ES + RT | 6 |
| 08816 | 1 | EAW + ES + RT | 7 |
| 08817 | 1 | EAW + ES + RT | 8 |
| 08818 | 1 | EAW + ES + RT | 9 |
| 08819 | 1 | EAW + ES + RT | 10 |
| 08820 | 0 | EAW + delayed-ES + noRT | 10C |

### Example 2: Pre-Implant Imaging, Antimicrobial Preparation, Temporary Implantation, and Permanent Implantation of Epidural Stimulation System

This Example describes pre-implant imaging, antimicrobial preparation, the two-step implantation procedure (temporary implantation followed by permanent implantation), perioperative monitoring, and postoperative recovery procedures.

Epidural stimulation system: An implanted ES system (Intellis Epidural Stimulator, Medtronic, Minneapolis, USA) was used to electrically stimulate the lumbosacral enlargement.

Antimicrobial decolonization: Prior to both temporary and permanent procedures, chlorhexidine soap skin cleanser (Hibiclens®) and mupirocin 2% ointment (Bactroban®) were provided for seven days to reduce bacterial colonization of participant skin and nares.

Preoperative procedures: Prior to operating room entry, participant consent was obtained, and an anesthesia preoperative evaluation was conducted. In a rare event when opioids were prescribed for more than five days, monitoring occurred according to clinical recommendations.

Magnetic resonance imaging (MRI): Prior to implantation, participants underwent MRI of the spine to evaluate spinal cord damage and ensure patency for implantation procedures. A General Electric 3.0 T Signa Discovery MR750 scanner was used with a two-dimensional fast spin echo sequence (slice thickness 5 mm; slice spacing 10 mm; field of view 200×200 mm²; matrix 320×224; repetition time 9015 ms; echo time 104 ms). Fifteen axial images were acquired using a two-dimensional fast relaxation fast spin echo sequence (slice thickness 3 mm; slice spacing 4.5 mm; field of view 360×360 mm²; matrix 512×512; repetition time 2939 ms; echo time 95 ms). Lesion hyperintensity was segmented on each axial slice followed by segmentation of the surrounding spinal cord using OsiriX MD (Pixmeo SARL, Bernex, Switzerland). Axial damage ratio was calculated as Arealesion/Areacord for each slice and the largest ratio was selected per participant. Lesion length was measured on the midsagittal T2-weighted image from the most cranial to most caudal ends of lesion hyperintensity.

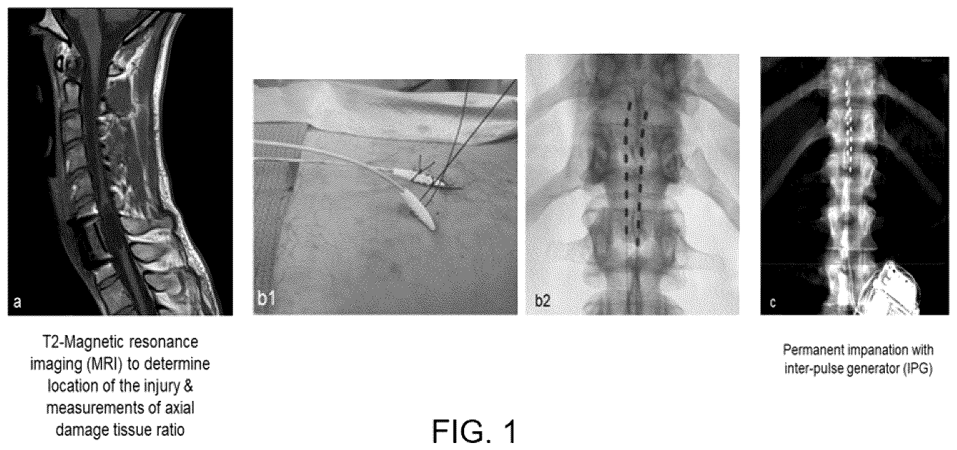

Temporary implantation: Temporary implantation was conducted to ensure proper lead placement and confirm activation of lumbosacral segments prior to proceeding with permanent implantation. Two 14-gauge epidural needles were used to access the epidural space using the loss-of-resistance technique with X-ray assistance. Two 8-electrode lead arrays were implanted utilizing fluoroscopic guidance over spinal segments T10-L2. Leads were inserted in the epidural space and stimulation configurations necessary to evoke motor potentials were tested as indicated by visible motor contractions of paralyzed muscles. Posterior epidural lead placement was confirmed by live fluoroscopy while the leads were guided to either side of the midline. Lead position was optimized after motor stimulation was confirmed. After lead placement, needles were removed, and electrodes were secured to the skin with tape and glue to limit migration. Spinal mapping was conducted for 3-5 consecutive days beginning on either the same or the following day. FIG. 1 (panels b1 and b2) depicts temporary implantation and external anchoring.

Permanent implantation and recovery: Seven days after temporary implantation, two 8-electrode arrays of Vectris leads were implanted in an operating room. Phase 1 of permanent implantation included establishing an IV line and maintaining ASA monitors including noninvasive blood pressure measured every five minutes, continuous EKG, pulse oximetry, and end tidal CO₂ from a nasal cannula. Antibiotics (clindamycin 600-900 mg or cefazolin 2-3 grams) were used during implantation. Sedatives, analgesics, and anesthetics were titrated when sedation was required. Using X-ray guidance, two 14-gauge epidural needles were used with loss-of-resistance technique to access the epidural space (FIG. 1, panel c).

Lead configurations and stimulation parameters necessary to evoke motor potentials were re-tested. An incision was made in the lower back or buttock, and a pulse generator was placed in a tissue pocket between muscle and skin. Leads were threaded under the skin to the pocket and connected to a Medtronic Intellis battery. Following hemostasis, the wound was closed in 2-3 layers, dermabond and occlusive dressing were applied, and tape was placed over the wound. A belly band was provided for comfort. Pain medicine was typically prescribed for three days. Recovery was typically completed at seven to ten days when bandages were removed. Each participant was examined three times in the first month for wound check (one week), dressing change (first week and second week), and reprogramming (fourth week).

### Example 3: Spinal Segmental Mapping, Recruitment Curves, and Stimulation Parameter Selection for Standing and Stepping

This Example describes spinal segmental mapping following implantation, including identification of stimulation parameters and configurations to activate targeted lower extremity muscle groups and joint movement patterns.

Mapping schedule and objectives: Following both temporary and permanent implantation, participants were scheduled for three to four days of spinal segmental mapping. Mapping identified stimulation parameters (frequency, amplitude, pulse duration), electrode polarity (cathodes versus anodes), and the number of channels required to evoke desired muscle contractions and joint movement patterns (hips, knees, ankles).

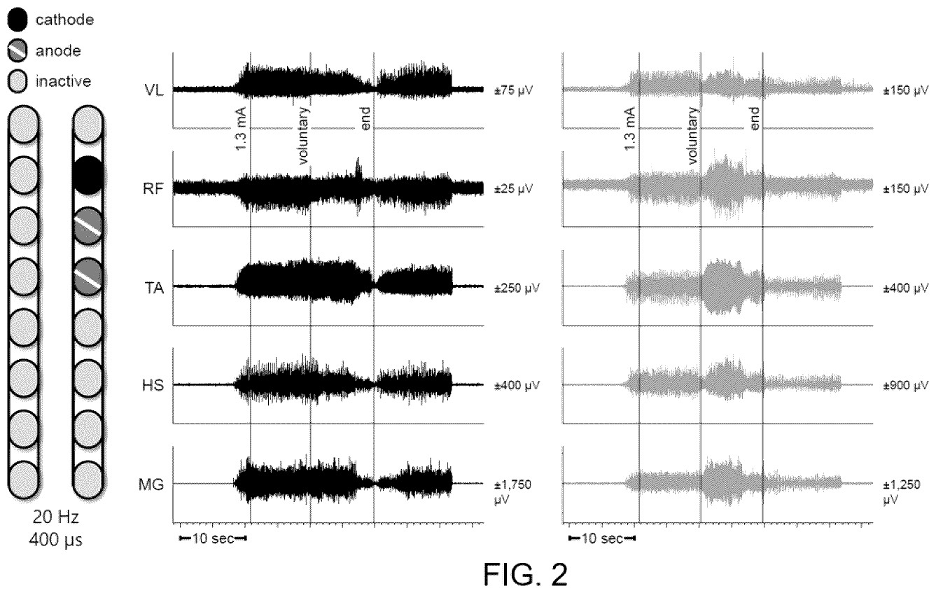

FIG. 2 shows an example of spinal mapping plots which were used to generate recruitment curves. Left leg muscles are depicted in black, right leg muscles in gray. Both plots occurred simultaneously but have been separated to better visualize different effects of voluntary effort between legs. At 1.3 mA, bilateral muscle activity was induced. However, when the participant volitionally attempted to flex his right leg, muscle activity decreased in some left leg muscles (VL, RF, HS) while increasing or bursting in right leg muscles (RF, TA, HS, MG). Hz=hertz (stimulation frequency); μs=microseconds; L=left; R=right; VL=vastus lateralis; RF=rectus femoris; TA=tibialis anterior; HS=hamstrings; MG=medial gastrocnemius; sec=seconds; uv=microvolts.

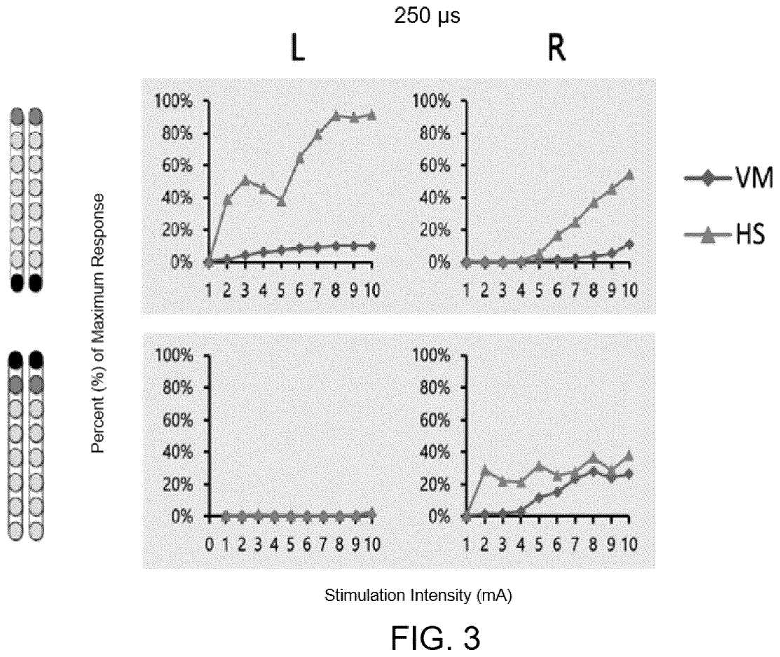

Target mapping and parameter ranges: Target mapping was used to identify stimulation configurations and parameters for different muscle groups. Frequencies of 2, 15, and 30 Hz and pulse durations of 250-1000 μs were tested. The minimum current (mA) necessary to evoke contraction was selected and monitored over the trial. FIG. 3 shows two exemplary recruitment curves at different cathode/anode activations, for left and right legs, showing percent response at a given stimulation intensity at the vastus medialis (VM) muscle of the quadriceps and hamstrings muscle (HM).

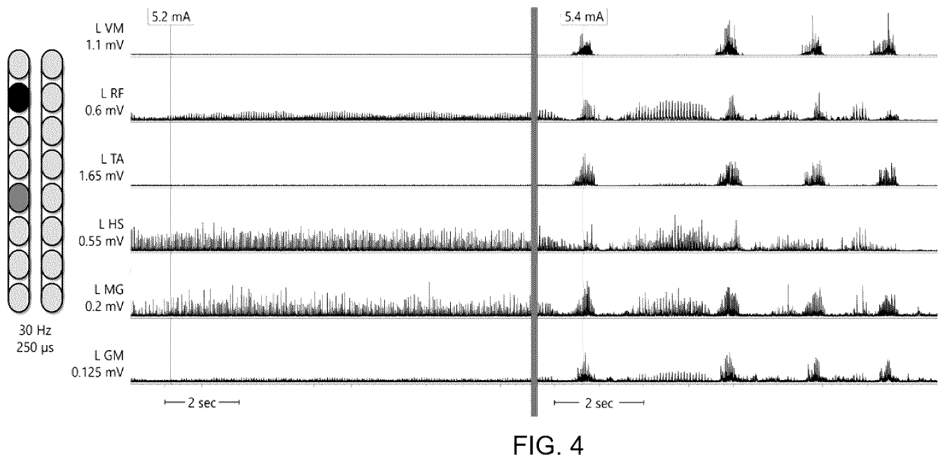

Volitional movement trials with stimulation off/on: From supine position, EMG electrodes were attached, and participants were asked to make movements including wiggling the big toe, ankle dorsiflexion, or moving the entire leg into flexion followed by extension, first with ES off and then with ES on. Up to 3-4 electrodes per channel were used to identify electrode combinations with stimulation parameters that evoked targeted responses. Participants then rested for two to three days and were trained to use the ES controller. FIG. 4 shows an example plot of rhythmic EMG activities of different muscle groups after setting the amplitude at 5.2 and 5.4 mA to be used in practicing stepping and locomotion at higher frequencies during either EAW or overground locomotion. Hz=hertz (stimulation frequency); μs=microseconds; L=left; R=right; VL=vastus lateralis; RF=rectus femoris; TA=tibialis anterior; HS=hamstrings; MG=medial gastrocnemius.

Recruitment curves for standing: For the first six months, the target was to achieve overground standing. Recruitment curves for tonic extensor activity in supine were developed using pulse durations of 250, 500, and 1000 μs; amplitude 1-10 mA; frequency 2 Hz. Recruitment curves were established using five configurations. Configurations yielding the highest extensor activity in supine were refined in standing at 10-40 Hz to determine standing configurations. Configurations for rhythmicity and stepping: Configurations intended to elicit rhythmicity in supine were determined (FIG. 4) and tested during EAW and stepping/locomotion, where possible, using parallel bars or a standing frame.

### Example 4: Intervention Procedures: Exoskeleton-Assisted Walking, Task-Specific Training, Resistance Training, and Passive Movement Control Procedures

This Example describes the intervention procedures implemented over the rehabilitation period, including exoskeleton-assisted walking, task-specific training, resistance training for the RT arm, and passive movement procedures for the control arm.

Exoskeleton-assisted walking (EAW): Prior to training, a research team member assisted the participant into the exoskeleton beginning with distal shoe support and proceeding proximally toward the trunk. Walking software was adjusted and progressed based on individual needs. Straps were adjusted to avoid excessive tightness to reduce risk of autonomic dysreflexia. EAW was scheduled three days per week for twelve months. Training began in first-step mode until participants achieved anterolateral weight shifting sufficient for stepping. Participants progressed to pro-step+mode starting with a standard roller walker and then to Canadian crutches in approximately four weeks. If less assistance was required, adaptability mode was used to gradually lower assistance based on walking performance. Assistance ranged from 0-100%. Once comfortable using Canadian crutches (~4 weeks), participants were encouraged to activate ES; training began at 100% assistance and support was reduced in 5% increments as tolerated until reaching the lowest assistance level possible. Assistance reduction decisions were based on ability to complete 80% of steps over a 10-meter distance without cueing. After dropping assistance, the exoskeleton allowed 2.4 seconds before passively moving the limb using a slow mode feature. Audible beeps differentiated active participant stepping versus passive exoskeleton stepping. Two buzzers cued weight shifting prior to stepping. With a trained research assistant providing posterior support, participants completed 60-90 minutes per session including fitting, rest, and walking. Vital signs (heart rate, blood pressure, oxygen saturation) were monitored every five minutes.

Task-specific training: Following permanent implantation, participants engaged in 60 minutes of task-specific training three times per week focusing on sit-to-stand activity and Romanian deadlift movements for trunk control using a standard walker or standing frame. The standing frame facilitated focusing on hip extension and locking of hip joints compared to combined hip and knee demands using a walker or parallel bars. Training patterns were alternated regularly. Task-specific training was conducted before or after exoskeleton training on the same day after a 60-minute rest period. Two or more research assistants provided support. Blood pressure was continuously monitored, and rest periods of 3-5 minutes were provided between attempts.

Resistance training (EAW+ES+RT group): Resistance training was performed twice weekly for six months, including open-chain training for the first twelve weeks and closed-chain training for the subsequent twelve weeks. Open-chain training used surface neuromuscular electrical stimulation (NMES) applied to knee extensors with ankle weights. Ankle weights were not applied in the first week to ensure full knee extension against gravity. Weight was increased by 2 pounds per week with the criterion that full knee extension was achieved before further increase. NMES was applied via two 8×10 cm² adhesive carbon electrodes (Uni-Patch, Wabasha, MI, USA) placed over knee extensors. Current was manually increased in 5-second intervals until knee extension was achieved against gravity, then gradually decreased to induce lengthening actions until full relaxation. The current (mA) producing full knee extension was recorded after each bout. Each session consisted of 4 sets of 10 repetitions over 45-60 minutes using a 5-second/5-second work/rest ratio, 3-minute rest between sets, 30 Hz stimulation, 450 μs pulses, and current sufficient to evoke full knee extension. Closed-chain training used implanted ES during gradual sit-to-stand exercise using a standard walker with the progression disclosed in Table 3. During the six months of RT, participants reported to the lab five times per week (3 sessions for EAW+ES and 2 sessions for RT).

Passive movement or passive stretching control procedure (EAW+delayed ES+noRT group): A research assistant supported the leg proximal to the ankle joint and moved it from 90 degrees knee flexion to full extension. The leg was maintained in extension for 5 seconds and returned to flexion for 5 seconds. Ten repetitions were performed on the right leg followed by ten on the left leg for a total of 4 sets×10 repetitions. Training was conducted five times per week during the final six months to balance the design, as described in Table 2.

TABLE 2
The progression of the closed chain resistance training via using implanted epidural stimulation.
Progression

| Both feet flat on the floor with | Both feet flat on the floor with | Both feet flat on the floor with |
|---|---|---|
| the knees and hips flexed > 90- | the knees and hips flexed = to | the knees and hips flexed < 90 |
| degrees | 90-degrees | degrees |
| 4 sets of 10 reps | 4 sets of 10 reps | 4 sets of 10 reps |

### Example 5: Motor, Locomotor, and Neurophysiologic Measurements and Data Processing

This Example describes locomotor, EMG, neurophysiologic, overground ambulation, and torque measurements, including disclosed acquisition and processing procedures.

10-meter EAW speed, unassisted steps, and EMG pattern: Walking time and speed were determined using a 10-meter walk test using a walker or crutches prior to implantation and every six months after implantation. The test was repeated with ES off and ES on. During each 10-meter test, the participant walked between two cones set 10 meters apart. A researcher used a counter to determine the number of unassisted steps (without audible cueing) and walking speed. Bilateral EMG (Delsys Inc., Massachusetts, USA) was recorded from muscles including soleus, medial gastrocnemius, tibialis anterior, medial hamstrings, quadriceps, and gluteus maximus using bipolar surface electrodes with fixed inter-electrode distance. ES artifacts were minimized using a 4th order Butterworth bandpass filter (20-450 Hz) using custom MATLAB scripts (MATLAB 2015a, The MathWorks Inc., Natick, MA). Filtered continuous data were segmented into individual strides using the vertical component of an accelerometer embedded within EMG sensors; this acceleration signal was lowpass filtered (4th order Butterworth, 3 Hz). Strides were segmented using an anteroposterior gyroscopic signal embedded within the gastrocnemius EMG sensor; each stride was segmented into stance and swing phases based on this signal. EMG RMS envelopes were calculated using a moving window of 150 samples for 10 strides. Peak filtered EMG values across the entire stride, stance phase, and swing phase were identified and averaged over the number of strides completed during each assistance level.

Neurophysiological assessments: One week prior to intervention (week 1) and during post-interventions 1 and 2, segmental sensorimotor reflexes were studied by recording H-reflexes and nociceptive reflexes; spinal motoneuron excitability was assessed by recording F-waves. Soleus H-reflex was recorded (20 Hz-20 kHz bandpass) with surface EMG on the soleus motor point in seated posture. Electrical stimulation (1-ms duration pulses) was delivered at 0.5 Hz using a constant current stimulator (DS7AH, Digitimer Ltd.) to the tibial nerve at the popliteal fossa via monopolar electrode; stimulus intensities were increased to obtain H-max, and amplitude and latency of H-max were measured. Nociceptive flexion reflex was recorded from biceps femoris (20 Hz-20 kHz bandpass). Electrical stimulation (train of five 1-ms pulses with inner frequency 300 Hz) was delivered to the sural nerve at the ankle via bipolar electrode; reflex threshold was determined by titrating intensity until stable reflex response with latency 80-180 ms; three stimuli were delivered at 20% above threshold. Motor F-waves were recorded from abductor hallucis (20 Hz-20 kHz bandpass) during tibial nerve stimulation at the ankle via bipolar electrode (0.2-ms pulses at 0.5 Hz). F-wave threshold and M-max threshold were recorded; ten stimuli were delivered at approximately 20% above M-max threshold.

Overground ambulation without exoskeleton: During post-interventions 1 and 2, overground ambulation without wearing the exoskeleton was tested using a zero-gravity gait system (Bioness System) during 10-meter walking distance with a standard walker equipped with sensors to determine weight bearing on the arms. Unassisted steps and walking speed were determined.

Peak torque and spasticity: Peak torque was evaluated using a Biodex isokinetic dynamometer (Shirley, NY). Participants were seated with trunk-thigh angle and knee-thigh angle at 90 degrees and secured with shoulder harnesses and hip belt. The dynamometer axis was aligned to the anatomical knee axis and lever arm attached 2-3 cm above the lateral malleolus. Passive tension of the right knee extensors was measured at 5, 30, 60, 90, 180, and 270 degrees/sec as an index of spasticity. Isometric torque was measured using current amplitudes 50 and 100 mA at frequency 30 Hz and pulse duration 450 μs. Isokinetic torque was measured at 60, 90, and 180 degrees/sec using the same stimulation protocol. Following implantation (post measurements 1 and 2), participants were asked to kick as strong as possible to measure torque generated at the knee joint with ES on. Each condition was tested with ES on and off to compare effects on torque generation and spasticity.

Walking Index for Spinal Cord Injury II (WISCI II): Walking performance was scored by selecting descriptors applicable to current walking performance and assigning the highest level at which the participant was safe as judged by a therapist. Comfort level and any nonstandard devices were documented. WISCI II was recorded at baseline and post measurement 2; change was calculated as Interval WISCI minus Baseline WISCI.

### Example 6: Cardio-Metabolic, Autonomic, Body Composition, Oxygen Consumption, and Muscle Oxygenation Measurements

This Example describes cardio-metabolic and autonomic measurements, body composition measurements, oxygen uptake testing during EAW, near-infrared spectroscopy measurements, and tilt-table testing with ES off and on.

Blood pressure and oxygen uptake (VO₂) during EAW: One week prior to intervention (week 1) and at post-measurements 1 and 2, VO₂ was measured using a COSMED K4b2 (COSMED USA, Chicago, IL) portable metabolic unit during a 6-minute walking test while wearing the exoskeleton. The protocol included 5 minutes sitting, 3 minutes standing, 6 minutes walking, 3 minutes standing, and 5 minutes sitting. After calibration, subjects wore a mask to monitor VO₂ and VCO₂, including 3-minute acclimation phases in sitting and standing. VO₂ and VCO₂ were monitored throughout walking to determine total energy expenditure using the Weir equation. A 5-minute recovery phase was recorded post-walk. Heart rate was monitored via Polar HR monitor and recorded every 30 seconds. Blood pressure was recorded continuously before exercise, every 2 minutes during EAW, and for 5 minutes during recovery using COSMED 740.

Body mass index (BMI): After voiding, participants were weighed on a wheelchair scale to obtain combined participant+wheelchair mass (kg-1). Participants transferred to a mat and wheelchair mass alone was obtained (kg-2). Participant mass was calculated as (kg-1)-(kg-2). Height was determined in supine using head and heel boards and measuring distance in centimeters. BMI (kg/m²) was calculated as mass (kg)/height² (m²).

Dual energy X-ray absorptiometry (DXA): DXA scans were performed using iLunar DXA (Lunar Inc., Madison, WI) after 20 minutes rest. Scans were performed and analyzed by a certified operator. Total and regional lean mass were determined. Disclosed prediction equations were applied: Muscle Mass MRI-WM=0.952 (LM_DXA)−0.073 (r²=0.90, SEE=0.23 kg, P<0.0001) and MMMRI-ABS=0.824 (LM_DXA)+0.018 (r²=0.82, SEE=0.28 kg, P<0.0001).

Basal metabolic rate (BMR) and biomarkers: After an overnight fast for 10-12 hours, participants were kept in a dark room for 20 minutes and BMR was measured using a canopy system and portable COSMED. Following BMR testing, fasting lipid profiles were assessed from 10 mL blood collected via indwelling catheter and analyzed by standard procedures. Total testosterone was measured by radioimmunoassay. Plasma IGF-I, IGFBP-3, and brain-derived neurotrophic factor concentrations were measured by immunoluminometric assay and radioimmunoassay. Inflammatory biomarkers (CRP, IL-6, TNF-α) were determined using ELISA kits. For oral glucose tolerance testing (OGTT), participants consumed 75 g glucose solution and blood was drawn at 30, 60, 90, 120, 150, and 180 minutes; duplicate samples were processed with EDTA and stored at −70 °C until analysis; plasma glucose was measured by glucose oxidase method and insulin by commercial radioimmunoassay.

Near-infrared spectroscopy (NIRS): Oxygenated and deoxygenated hemoglobin in gastrocnemius were measured using a portable NIRS unit (Portamon, Artinis Medical Systems). The probe was placed longitudinally along the belly of the right gastrocnemius ~3-5 cm below the popliteal fossa and secured by Velcro strap. A blood pressure cuff (Hokanson SC-10D, Hokanson, Inc., Bellevue, WA) was placed proximal to the probe; the cuff was controlled by Hokanson E20 system set to 250 mmHg. Resting muscle oxygen consumption was measured by 30-second cuff inflation. Physiological calibration (ischemic calibration) established 0% saturation during occlusion and 100% upon release. Muscle oxygen consumption (mVO₂) was determined from slope of HHb signal during walking compared to baseline sitting. Measurements were conducted during the 6-minute walk test in conjunction with metabolic measurements.

Tilt table test for autonomic function: Participants rested supine for 5 minutes while a 5-lead ECG collected beat-by-beat heart rate data and a finger cuff collected beat-by-beat continuous blood pressure. The tilt table was raised to 60 degrees and maintained up to 10 minutes with continued monitoring. The table was lowered before 10 minutes if systolic blood pressure dropped by more than 40 mmHg from baseline or upon participant request. Participants then completed a 5-minute supine recovery. The protocol was repeated with ES on after a 30-minute washout; ES was turned on 3-5 minutes in supine prior to tilt. Outcomes derived included duration of tilt, low-frequency and high-frequency power during tilt, and cardiovascular responses at initiation and during tilt.

### Example 7: Urodynamic Testing Procedures and Outcomes; Recruitment Outcomes

This Example describes urodynamic testing, the standardized fill protocol, outcome definitions, recruitment/disposition outcomes, and disclosed trial results statements presented within the disclosure.

Urodynamic testing: Urodynamics were performed to evaluate bladder function. Individuals managing bladder function with clean intermittent self-catheterization were recruited. A 3-day frequency-volume chart was completed to provide baseline bladder capacity information. At baseline urodynamics, initial fill rate was set to 10% of maximum catheterized volume per minute to standardize fill rates. Cystometric capacity was defined as the volume at which involuntary contraction with leakage occurred during the first fill, and 10% of cystometric capacity was used for subsequent fills in which urodynamic data were collected.

Urodynamic outcome measures included: (1) development of volitional voiding after a permission-to-void command; (2) time from permission-to-void to onset of voluntary voiding; (3) voiding efficiency defined as voided volume divided by (infused volume+post-void residual); (4) suppression of involuntary contractions quantified by change in peak pressure; (5) suppression of urinary urgency; (6) alterations of bladder sensation thresholds including first sensation of filling, first desire to void, and strong desire to void; (7) assessment of bladder outlet obstruction using ICS nomogram and obstruction index PdetQmax−2 Qmax; and (8) bladder contractility index PdetQmax+5 Qmax.

Recruitment and disposition outcomes: Table 4 lists example participant IDs and disposition categories including completed, withdrew, and screen-failed, along with sex, level of injury, time since injury (years), AIS classification, and injury classification. C indicates completed; X indicates withdrawn or screen-failed.

TABLE 3
The number of persons who enrolled, completed, or withdrawn from the study.

| | | | | | | | TSI | | |
|---|---|---|---|---|---|---|---|---|---|
| Subject ID | TT + LPWS/NMES | Base 1 | >Int. | >Int. 2 | Sex | LOI | (yrs.) | AIS | Class |
| 0881 | EAW + ES + RT | C | C | C | M | C8 | 5 | A | Tetraplegia |
| 0882 | EAW + ES + RT | C | C | X | M | T11 | 7 | B | Paraplegia |
| 0883 | EAW + delayed-ES + noRTC | C | C | C | M | T6 | 12 | A | Paraplegia |
| 0884 | EAW + ES + RT | C | C | C | M | T4 | 24 | A | Paraplegia |
| 0885 | Withdrew | X | X | X | M | T7 | 2 | B | Paraplegia |
| 0886 | Withdrew | X | X | X | M | T9 | 3 | A | Paraplegia |
| 0887 | Screen failed | X | X | X | M | T3 | 2.5 | A | Paraplegia |

Restoration of locomotion with ES and EAW was described as resulting in a reduction of psychosocial, cardiovascular, metabolic, bladder parameters and socioeconomic burden. Addition of the resistance training paradigm was described as augmenting outcomes of ES on motor function. Percutaneous ES was described as feasible and safe for restoration of motor function and as implementable with task specific training including EAW and resistance training.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

What is claimed is:

1. A method of implanting and configuring an epidural spinal cord stimulation system for a subject with spinal cord injury, the method comprising:
temporarily implanting at least one electrical stimulation lead into an epidural space of the subject;
delivering epidural electrical stimulation through the temporarily implanted lead;
performing spinal segmental mapping during the temporary implantation by varying at least one electrode configuration or stimulation parameter and assessing motor responses of one or more lower extremity muscles of the subject;
determining, based on the spinal segmental mapping, a target epidural location or stimulation configuration associated with a desired motor activation pattern;
removing the temporarily implanted lead; and
permanently implanting at least one electrical stimulation lead into the epidural space of the subject at the target epidural location determined from the spinal segmental mapping.

2. The method of claim 1, wherein the spinal segmental mapping comprises recording electromyographic (EMG) signals from a plurality of lower extremity muscles.

3. The method of claim 1, wherein the spinal segmental mapping identifies stimulation configurations that produce tonic motor activation associated with leg flexion or knee extension.

4. The method of claim 1, wherein the spinal segmental mapping identifies stimulation configurations that produce rhythmic motor activity associated with stepping or locomotion.

5. The method of claim 1, wherein determining the target epidural location comprises selecting a spinal segment corresponding to a lumbosacral enlargement.

6. The method of claim 1, wherein permanently implanting comprises implanting a plurality of epidural leads in a staggered configuration spanning multiple vertebral levels.

7. The method of claim 1, further comprising electrically coupling the permanently implanted lead to an implantable pulse generator configured to deliver stimulation according to the determined stimulation configuration.

8. A method of selecting an epidural implantation site for permanent spinal cord stimulation, the method comprising:
temporarily placing at least one electrical stimulation lead within an epidural space of a subject;
delivering epidural stimulation through the temporarily placed lead using a plurality of electrode configurations or stimulation parameter sets;
evaluating motor responses of the subject's lower extremities during the epidural stimulation;
mapping epidural locations to corresponding to characteristics of motor responses of the subject; and
selecting, based on the mapping, an epidural implantation site for permanent placement of a spinal cord stimulation lead.

9. The method of claim 8, wherein evaluating motor responses comprises observing visible muscle contractions in paralyzed lower extremity muscles.

10. The method of claim 8, wherein evaluating motor responses comprises analyzing electromyographic (EMG) recruitment curves.

11. The method of claim 8, wherein mapping epidural locations includes distinguishing between tonic motor responses and rhythmic motor responses.

12. The method of claim 8, wherein the selecting step further comprises determining whether the subject is a candidate for permanent implantation.

13. The method of claim 8, wherein the temporary placement is maintained for multiple days to perform repeated mapping sessions.

14. The method of claim 8, further comprising permanently implanting a stimulation lead at the selected epidural implantation site.

15. A method of treating a subject with spinal cord injury, the method comprising:
temporarily implanting at least one epidural stimulation lead into an epidural space of the subject;
delivering epidural stimulation through the temporarily implanted lead while varying stimulation parameters to evoke motor responses of the subject's lower extremities;
mapping motor responses of the subject to stimulation parameters and epidural lead locations;
removing the temporarily implanted lead;
permanently implanting at least one epidural stimulation lead based on the mapping; and
delivering epidural stimulation through the permanently implanted lead to facilitate lower extremity motor function.

16. The method of claim 15, wherein delivering epidural stimulation through the permanently implanted lead is performed while the subject performs standing or walking training.

17. The method of claim 15, wherein delivering epidural stimulation through the permanently implanted lead is performed while the subject performs exoskeleton-assisted walking.

18. The method of claim 15, wherein the mapping identifies stimulation parameters that reduce stimulation amplitude required to evoke motor responses.

19. The method of claim 15, wherein the mapping is performed while the subject is in at least a supine position and a standing position.

20. The method of claim 15, wherein the treatment improves voluntary motor control, assisted stepping performance, or both.
